(19) United States
(12) Patent Application Publication
(10) Pub. No.: US 2025/0160729 A1
(43) Pub. Date: May 22, 2025

(54) SYSTEM AND METHOD FOR ASSESSING NEUROLOGICAL FUNCTION

(71) Applicant: Cambridge Cognition Limited, Cambridge, Cambridgeshire (GB)

(72) Inventors: Nicholas Theodore TAPTIKLIS, Cambridgeshire (GB); Francesca Kathleen CORMACK, Cambridgeshire (GB); Matthew ALLEN, Cambridgeshire (GB); Alex KAULA, Cambridgeshire (GB)

(73) Assignee: Cambridge Cognition Limited, Cambridge, Cambridgeshire (GB)

(21) Appl. No.: 18/832,273

(22) PCT Filed: Jan. 24, 2023
(86) PCT No.: PCT/GB2023/050150

§ 371 Date: Jul. 23, 2024

(30) Foreign Application Priority Data
Jan. 24, 2022 (GB) 2200859.3

(51) Int. Cl. A61B 5/00; G10L 15/10; G10L 15/18

(52) U.S. Cl. CPC A61B 5/4088; A61B 5/4803; A61B 5/7246; A61B 5/7282; G10L 15/10; G10L 15/1815

(57) ABSTRACT

According to a first aspect of the disclosure there is provided a system for assessing the neurological function of a subject, comprising: a database storing a plurality of word groups, said word groups comprising two or more words; a word group memorability determining part for determining memorability of the word groups based on one or more characteristics of the words in the word groups; a test set generating part for generating a plurality of sets of word groups, based on the memorability of the word groups; and a test delivering part for delivering a test to the subject in the form of at least one set of the plurality of sets of word groups for recall by the subject.

### TECHNICAL FIELD

The present disclosure relates to methods and systems for assessing the neurological function of a subject. In some examples the neurological function may relate to memory, e.g. episodic memory.

### BACKGROUND ART

The Verbal Paired Associates (VPA) test is one of the most widely used instruments for assessing explicit episodic memory performance, sensitive to hippocampal decline associated with Alzheimer's disease. In administering the assessment, an examiner will read a list of word pairs for the subject to remember, and then one by one ask the subject to recall which target word was paired with each initial word from the list.

Each set of word-pairs can only be used once per subject. To facilitate repeat testing, a few additional sets of word pairs have been produced, with the difficulty/memorability of pairs considered a function of the semantic relatedness of the words used. Traditional sets of word-pairs contain a mix of easy pairs (semantically related words such as “Fruit” & “Apple” or “Baby” & “Cries”) and hard pairs (unrelated words such as “Cabbage” & “Pen”).

However, it is difficult to perform high-frequency, repeat testing of subjects based on the present methodology. Further, tests are administered and result recorded by examiners, often different each time, which introduces problems with consistency and objectivity.

The present disclosure aims to at least partially address some of the problems described above.

### SUMMARY OF THE INVENTION

According to a first aspect of the disclosure there is provided a system for assessing the neurological function of a subject, comprising: a database storing a plurality of word groups, said word groups comprising two or more words; a word group memorability determining part for determining memorability of the word groups based on one or more characteristics of the words in the word groups; a test set generating part for generating a plurality of sets of word groups, based on the memorability of the word groups; and a test delivering part for delivering a test to the subject in the form of at least one set of the plurality of sets of word groups for recall by the subject.

Optionally, the one or more characteristics comprises a measure of semantic relatedness between words in the word group, a higher semantic relatedness being associated with a higher memorability. Optionally, sematic relatedness is measured by representing words as vectors using a machine learning algorithm and measuring cosine distance between said vectors.

Optionally, the one or more characteristics comprises a measure of word-frequency of words in the word group, a higher word-frequency being associated with a higher memorability. Optionally, word-frequency is attributed to a word based on a look-up table comprising pre-stored a word-frequency for each word.

Optionally, the one or more characteristics comprises a measure of word-concreteness of words in the word group, a higher word-concreteness being associated with a higher memorability. Optionally, word-concreteness is attributed to a word based on a look-up table comprising pre-stored a word-concreteness for each word.

Optionally, the one or more characteristics comprises the position of a word in the word group.

Optionally, the system further comprises a phonetic similarity determining part for determining the phonetic similarity between words in the word groups, wherein the test set generating part generates the plurality of sets of word groups based on the phonetic similarity between words in the word groups. Optionally, the phonetic similarity is below a predefined threshold, such that the words are phonetically dissimilar. Optionally, phonetic similarity is measured by representing the words phonetically and measuring an edit distance between said phonetic representations.

Optionally, the test delivering part delivers the at least one set of word groups aurally.

Optionally, the system further comprises: a response receiving part for receiving a response from the subject attempting to recall the delivered at least one set of word groups.

Optionally, the response receiving part is configured to receive spoken responses.

Optionally, the system further comprises a speech analysis part for determining a textual response from the spoken response.

Optionally, the system further comprises: a response assessment part for comparing the response from the subject to the delivered at least one set of word groups. Optionally, the response assessment part compares a textual response from the subject to the delivered at least one set of word groups.

Optionally, the system is configured to deliver at least two tests with predetermined time intervals between said tests, wherein the sets of word groups of the delivered tests are different and the memorability of the sets of word groups of the delivered tests satisfy a predetermined relationship. Optionally, the predetermined relationship is that the tests have the same or similar memorability. Optionally, the predetermined relationship is that consecutive tests have an incrementally lower memorability or an incrementally higher memorability.

According to a second aspect of the disclosure there is provided a system for assessing the neurological function of a subject, comprising: a database storing a plurality of word groups, said word groups comprising two or more words; a phonetic similarity determining part for determining the phonetic similarity between words in the word groups, a test set generating part for generating a plurality of sets of word groups based on the phonetic similarity between words in the word groups; and a test delivering part for delivering test to the subject in the form of at least one set of the plurality of sets of word groups for recall by the subject.

Optionally, the sets are generated such that the phonetic similarity between words is below a predefined threshold, such that the words are phonetically dissimilar.

Optionally, the system further comprises a word group generating part for generating the plurality of word groups from a base list of words and storing in said database.

According to a third aspect of the disclosure there is provided a computer implemented method for assessing the neurological function of a subject, comprising: storing a plurality of word groups, said word groups comprising two or more words; determining memorability of the word groups based on one or more characteristics of the words in the word groups; generating a plurality of sets of word groups, based on the memorability of the word groups; and delivering test to the subject in the form of at least one set of the plurality of sets of word groups for recall by the subject.

According to a fourth aspect of the disclosure there is provided a computer implemented method for assessing the neurological function of a subject, comprising: reading a plurality of word groups stored in a memory, said word groups comprising two or more words; determining memorability of the word groups based on one or more characteristics of the words in the word groups; generating a plurality of sets of word groups, based on the memorability of the word groups; and delivering test to the subject in the form of at least one set of the plurality of sets of word groups for recall by the subject.

According to a fifth aspect of the disclosure there is provided a computer implemented method for assessing the neurological function of a subject, comprising: storing a plurality of word groups, said word groups comprising two or more words; determining the phonetic similarity between words in the word groups, generating a plurality of sets of word groups based on the phonetic similarity between words in the word groups; and delivering test to the subject in the form of at least one set of the plurality of sets of word groups for recall by the subject.

According to a sixth aspect of the disclosure there is provided a computer implemented method for assessing the neurological function of a subject, comprising: reading a plurality of word groups stored in a memory, said word groups comprising two or more words; determining the phonetic similarity between words in the word groups, generating a plurality of sets of word groups based on the phonetic similarity between words in the word groups; and delivering test to the subject in the form of at least one set of the plurality of sets of word groups for recall by the subject.

According to a seventh aspect of the disclosure there is provided a computer system comprising at least one processor and at least one memory configured to implement assessing the neurological function of a subject: the at least one memory comprising a database configured to store a plurality of word groups, said word groups comprising two or more words; the at least one processor comprising a word group memorability determining part configured to determine memorability of the word groups based on one or more characteristics of the words in the word groups; the at least one processor comprising a test set generating part configured to generate a plurality of sets of word groups, based on the memorability of the word groups; and the at least one processor comprising a test delivering part configured to deliver test to the subject in the form of at least one set of the plurality of sets of word groups for recall by the subject.

According to a eighth aspect of the disclosure there is provided a non-transitory computer-readable medium storing code which, when executed by at least one processor of a computer system, causes the system to implement assessing the neurological function of a subject, comprising: reading at least one memory of the computer system comprising a database storing a plurality of word groups, said word groups comprising two or more words; a word group memorability determining part of the at least one processor determining memorability of the word groups based on one or more characteristics of the words in the word groups; a test set generating part of the at least one processor generating a plurality of sets of word groups, based on the memorability of the word groups; and a test delivering part of the at least one processor delivering a test to the subject in the form of at least one set of the plurality of sets of word groups for recall by the subject.

According to a ninth aspect of the disclosure there is provided a computer system comprising at least one processor and at least one memory configured to implement assessing the neurological function of a subject: the at least one memory comprising a database configured to store a plurality of word groups, said word groups comprising two or more words; the at least one processor comprising a phonetic similarity determining part configured to determine the phonetic similarity between words in the word groups, the at least one processor comprising a test set generating part configured to generate a plurality of sets of word groups based on the phonetic similarity between words in the word groups; and the at least one processor comprising a test delivering part configured to deliver a test to the subject in the form of at least one set of the plurality of sets of word groups for recall by the subject.

According to a tenth aspect of the disclosure there is provided a non-transitory computer-readable medium storing code which, when executed by at least one processor of a computer system, causes the system to implement assessing the neurological function of a subject, comprising: reading at least one memory comprising a database configured to store a plurality of word groups, said word groups comprising two or more words; a phonetic similarity determining part of the at least one processor determining the phonetic similarity between words in the word groups, a test set generating part of the at least one processor generating a plurality of sets of word groups based on the phonetic similarity between words in the word groups; and a test delivering part of the at least one processor delivering a test to the subject in the form of at least one set of the plurality of sets of word groups for recall by the subject.

### BRIEF DESCRIPTION OF THE FIGURES

Features and advantages of the disclosure are described in further detail below by way of non-limiting examples and with reference to the accompanying drawings in which:

FIG. 1 shows an example system according to the disclosure;

FIG. 2 shows a subject interacting with a device;

FIG. 3 shows a first example process; and

FIG. 4 shows a second example process.

### DETAILED DESCRIPTION

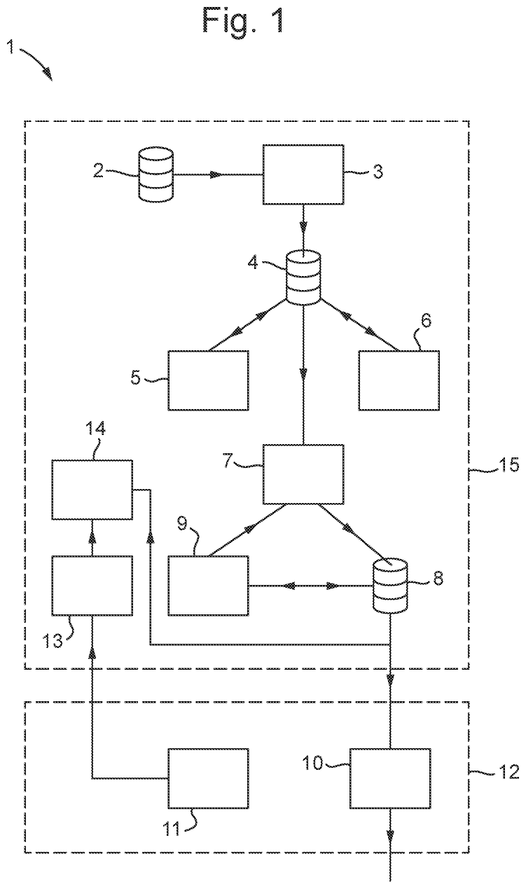

FIG. 1 shows an example system for assessing the neurological function of a subject, comprising: a database 2 storing a plurality of word groups, said word groups comprising two or more words; a word group memorability determining part 5 for determining memorability of the word groups based on one or more characteristics of the words in the word groups; a test set generating part 7 for generating a plurality of sets of word groups, based on the memorability of the word groups; and a test delivering part 10 for delivering a test to the subject in the form of at least one set of the plurality of sets of word groups for recall by the subject.

As shown in FIG. 1, an example system 1 according to the disclosure may comprise a first database 2 that stores a base list of candidate words for use in a recall test. The words may conform to general suitability characteristics, for example profanities, single-syllable words, very long words, overly technical or obscure words may not be included. The database may comprise any typical electronic data storage medium, such as a computer memory.

As shown in FIG. 1, the system 1 may comprise a word group generating part 3 for generating a plurality of word groups from the base list of words and storing the plurality of word groups in a second database 4. The word groups may comprise two or more different words from the base list. The word group generating part 3 may generate word groups consisting of two words, i.e. word pairs. However, the present disclosure is not limited to generating word pairs-word groups consisting of more than two words, e.g. word triples, or groups of fifteen words may be generated instead. The word group generating part 3 may form a part of one or more computer processors forming part of the system 1, which may be a computer system.

Preferably, the list of generated word groups consists of word groups having the same number of words. However, in some example systems, the list may comprise word groups with different numbers of words, i.e. of different lengths. Longer word groups may have a lower memorability than shorter word groups.

In an example system, the word group generating part may generate a list of word groups that comprises every possible combination of n words, n being the number of words in each group. In an example system, the list of generated word groups may consist of word groups wherein the combination of words in the word group is unique to each word group. For example, only one of the pairs “fruit, apple” and “apple, fruit” would be present in the generated list. In an alternative system, the list of generated word groups may comprise word groups consisting of the same words as another word group in the list but with the words in a different order.

As shown in FIG. 1, the system 1 may comprise a word group memorability determining part 5 for determining memorability of the word groups stored in the second database 4. Memorability may be a parameter that represents the relative difficulty of recalling the words in the word group. The memorability may be based on one or more characteristics of the words in the word groups. The memorability may also be stored in the second database 4. The word group memorability determining part 5 may form a part of one or more computer processors forming part of the system 1, which may be a computer system.

In an example, the one or more characteristics may comprise a measure of semantic relatedness between words in the word group, a higher semantic relatedness being associated with a higher memorability. Semantic relatedness relates to how similar the meaning of the words is. Sematic relatedness may be measured using word embeddings, e.g. by representing words as multi-dimensional vectors using a machine learning algorithm and measuring cosine distance between said vectors. For example, the algorithm GloVe: Global Vectors for Word Representation available at https://nlp.stanford.edu/projects/glove/may be used. The semantic relatedness may be derived from a language corpus, which may comprise, for example, hundreds of millions of words in use in natural language. The language corpus may comprise text from a wide range of genres (e.g. spoken, fiction, magazines, newspapers, and academic). The British National Corpus is an example of such a corpus.

In an example, the one or more characteristics may comprise a measure of word-frequency of words in the word group, a higher word-frequency being associated with a higher memorability. Word-frequency relates to how frequently a word occurs in the language, i.e. how common it is. Word-frequency may be attributed to a word based on a look-up table comprising pre-stored word-frequency for each word. This information may be part of the base list. Alternatively, the word-frequency may be derived from the language corpus, based on the number of instances of each word in the language corpus.

In an example, the one or more characteristics comprises a measure of word-concreteness of words in the word group, a higher word-concreteness being associated with a higher memorability. Word concreteness relates to whether a word is “concrete” or “abstract”. A concrete word may refer to tangible objects, materials or persons which can be easily perceived with the senses, for example. Word-concreteness may be attributed to a word based on a look-up table comprising pre-stored word-concreteness for each word. This information may be part of the base list.

Alternatively, word concreteness may be measured using word embeddings, e.g. by representing words as multi-dimensional vectors using a machine learning algorithm. In such an example, word concreteness could be derived using the machine learning algorithm. The word-concreteness may be derived from the language corpus.

In an example, the one or more characteristics may comprise the position of a word in the word group. Typically, words at the start or end of a sequence are more memorable. This can be taken into account when determining the overall memorability of a word group, e.g. by weighting other characteristics according to word position. For example, in the word group ‘metal-edition-driver-mixture’, the words ‘metal’ and ‘mixture’ may be more memorable than ‘edition’ and ‘driver’.

In an example, the one or more characteristics may comprise the position of a word group in a set. Typically, word groups at the start or end of a set, presented to a subject in sequence are more memorable. This can be taken into account when determining the memorability of a word-group, e.g. by weighting individual memorability according to word group position. For example, in the set below, ordered as the sequence would be delivered to the subject, the word groups ‘metal—leather’ and ‘virgin—emerge’ may benefit in memorability from their position in the list, whereas ‘mixture—combine’ and ‘suspend—resume’ may be rendered more difficult by virtue of their positions.
metal—leather
edition—publish
driver—formula
mixture—combine
suspend—resume
endorse—approve
ally—unite
virgin—emerge

In an example using the above characteristic, the memorability may be determined after a test set is generated.

In an example, memorability of a word group may be modelled based on one or more of the above characteristics and preferably all of the above characteristics. The word group memorability determining part 5 applies the model to the word groups to determine the memorability.

As shown in FIG. 1, the system 1 may comprise a phonetic similarity determining part 6 for determining the phonetic similarity between words in the word groups stored in the second database 4. Phonetic similarity relates to how similarly the words are pronounced. Words that are pronounced similarly may lead to confusion when delivering a test and when receiving a response, which can reduce testing efficacy. The phonetic similarity may also be stored in the second database 4. The phonetic similarity determining part 6 may form a part of one or more computer processors forming part of the system 1, which may be a computer system.

In an example, phonetic similarity may be measured by representing the words in a word group phonetically then measuring an edit distance between said phonetic representations. An edit distance may be the minimum number of changes to the phonetic representation required to change from one word to another. For example, a Levenshtein distance or Jaro-Winkler distance may be used.

In an example, phonetic similarly may be measured by machine learning. For example, using speech generation to generate audio for a word, then deep-learning based speech recognition to generate an embedded vector representation (e.g. Connectionist Temporal Classification tensor) of the sound, then measuring the cosine distance or similar between words. In some examples, actual spoken response may be used to refine the machine learning model, e.g. in the same way as speech generated audio for a word described above.

As shown in FIG. 1, the system 1 may comprise a test set generating part 7 for generating a plurality of sets of word groups for testing the subject. The test sets may be stored in a third database 8. The sets may comprise a predefined number of word groups. In an example, eight word pairs comprise a single test set. The test set generating part 7 may select word groups generated by the word group generating part 3, e.g. stored in the second database 4, and generate test sets based on one or more criteria. The test set generating part 7 may form a part of one or more computer processors forming part of the system 1, which may be a computer system.

In an example, the word groups may be selected to produce a set having a predefined compound memorability. The compound memorability may represent the memorability of the test set as a whole. In addition to the memorability of each word group within the set, the compound memorability may be determined based on one or more characteristics of the set of words groups. The compound memorability may be determined by a set memorability determining part 9 forming part of the word group memorability determining part 5.

In an example, the one or more characteristics of the set may comprise the position of a word group in the set. As described above, word groups at the start or end of a set, presented to a subject in sequence are more memorable. This can be taken into account when determining the overall memorability of a set, e.g. by weighting individual memorability according to word group position.

In an example, a plurality of test sets may be generated with each test set having the same, or similar, compound memorability. For example, the compound memorability of each set may be within a predefined range of compound memorability. In an example a test set may be generated to have a compound memorability that is the same as, or similar to, a previous test set for the same subject.

In an example, a plurality of test sets may be generated with each test set having incrementally increasing or decreasing compound memorability. In an example a test set may be generated to have a compound memorability that is incrementally higher, or incrementally lower, than a previous test set for the same subject.

In an example, the word groups forming a set may be selected to have a predefined memorability. For example, the memorability of each word group in the set may have the same, or similar, memorability. Alternatively, the memorability of each word group in the set may follow a predefined pattern, such as incrementally increasing or decreasing memorability.

In an example, the test set generating part 7 may generate sets of word groups such that the phonetic similarity between words in each word group forming the test sets is below a predefined threshold, such that the words are phonetically dissimilar. For example, a word group comprising phonetically similar words may not be selected and a word group comprising phonetically dissimilar words may be selected.

In an example, the test set generating part 7 may generate sets of word groups such that the phonetic similarity between words in different word groups forming the test sets is below a predefined threshold. For example, different word groups comprising phonetically similar words may not be selected and different word group comprising phonetically dissimilar words may be selected. The phonetic similarity of words in different word groups may be determined by the phonetic similarity determining part 6, e.g. by processing each combination or words in different word groups. In an example, phonetic similarity may only be determined at this stage.

In an example, the test set generating part 6 may generate a plurality of candidate test sets and then apply one or more of the above criteria to reject those test sets that do not satisfy the criteria and accept those sets that do satisfy the criteria.

By generating the test sets in the above described manner, high-quality, high-frequency repeat testing can be achieved.

As shown in FIG. 1 the system 1 may comprise a test delivering part 10 for delivering test to the subject in the form of at least one set of the plurality of sets of word groups for recall by the subject. In an example, one test set of word groups is used for each test.

In an example, the test delivering part 10 delivers a set of word groups for the subject to remember, and then may request the subject to recall which target word (or words) was paired with each initial word (known as a cue or prompt word) from a word group in the set. The test delivering part 10 may additionally deliver instructions to the subject for performing the test, such as “Please try to remember the following set of word-pairs, you will later be tested”.

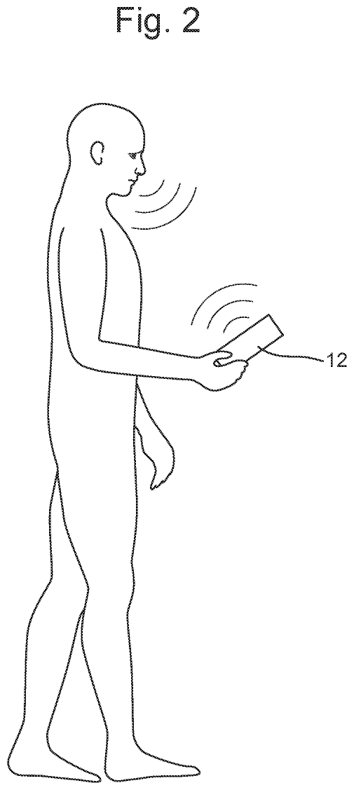

The test delivering part 10 may be provided on a device 12 such as a personal computer, tablet computer, smart phone, smart watch or the like. The device 12 may comprise hardware, such as a speaker or screen, for delivering a test. The test delivering part 10 may be formed by one or more computer processors forming part of the system 1, which may be a computer system. The test delivering part 10 may be configured to control the hardware, such as a speaker or screen, for delivering a test. A user may interact with the device 12 in the usual manner, via a user interface. The test delivering part 10 may be implemented by an application on the device 12. Interaction between the user and the device 12 is shown in FIG. 2.

In an example, the test delivering part 10 may deliver the at least one set of word groups aurally, e.g. via a speaker. In an example, the test delivering part 10 may deliver the at least one set of word groups visually, e.g. via a screen. In the case of visual delivery, the test may be read to the subject. The test delivering part 10 may be configurable between aural and/or visual delivery. By selecting phonetically dissimilar words, the comprehension of the sets by the subject may be increased. Word sets can be generated whereby the whole sets are optimised to minimise confusability between words, to the benefit of the test subject, particularly those who are hard of hearing.

As shown in FIG. 1, the system May 1 may comprise a response receiving part 11 for receiving a response from the subject attempting to recall the delivered at least one set of word groups. The response receiving part 11 may be configured to receive spoken responses, e.g. via microphone. The response receiving part 11 may be provided on the device 12. The device 12 may comprise hardware, such as microphone, for receiving and recording a response to a test. The response receiving part 11 may be formed by one or more computer processors forming part of the system 1, which may be a computer system. The response receiving part 11 may be configured to control the hardware, such as microphone, for receiving and recording a response to a test.

As shown in FIG. 1, the system may comprise a speech analysis part 13 to determine a textual response from the spoken response, e.g. by automatic speech recognition. By selecting phonetically dissimilar words, the accuracy of automatic speech recognition may be greatly increased. Word sets can be generated whereby the whole sets are optimised to minimise confusability between words, to the benefit of automated speech recognition systems. The speech analysis part 13 may form a part of one or more computer processors forming part of the system 1, which may be a computer system.

As shown in FIG. 1 the system may comprise a response assessment part 14 for comparing the response from the subject to the delivered at least one set of word groups. The response assessment part 14 may compare the textual response from the subject, e.g. determined by the speech analysis part 13, to the delivered at least one set of word groups. Based on this, the response assessment part 14 may determine a test score indicating the performance of the subject. The score may be communicated to the subject, or to their clinician, for example. The response assessment part 14 may form a part of one or more computer processors forming part of the system 1, which may be a computer system.

As shown in FIG. 1 each of the first database 2, word group generating part 3, second database 4, word group memorability determining part 5, phonetic similarity determining part 6, test set generating part 7, third database 8, set memorability determining part 9, speech analysis part 13, and response assessment part 14 may be provided by a remote server 15. The remote server 15 may be separate from any device 12 that may comprise the test delivery part 10 and/or the response receiving part 11. The databases 2, 4 and 8 may be provided by one or more computer memory units. The processing parts 3, 5, 6, 7, 9, 10, 11 13 and 14 may be provided by one or more computer processors.

In an example, the system 1 is configured to deliver at least two tests with predetermined time intervals between said tests, wherein the sets of word groups of the delivered tests are different and the memorability of the sets of word groups of the delivered tests satisfy a predetermined relationship. In an example, the predetermined relationship is that the tests have the same or similar compound memorability. In another example, the relationship is that consecutive tests have an incrementally lower memorability or an incrementally higher memorability. In a further example, the memorability of word groups may be dynamically determined on an individualised basis, whereby the individual's previous performance is used to select a group of words of predicted memorability for that individual.

In an example system, word lists and word groups may be generated in multiple different languages. Tests sets may be “translated” from one language to another based on equivalent memorability characteristics rather than literal translation. In other words, a translation of a base set in a first language would provide a translated set in a second language with the same, or similar, memorability characteristics as the base set. The system may comprise a base list in each language and/or derive characteristics based on a language corpus for each language. Accordingly, a single system may enable tests to be delivered in multiple languages.

In an example system, words may be selected based on an age of acquisition associated with each word (e.g. stored in the base list), i.e. when the word is first learned on average. By setting upper and/or lower thresholds on age of acquisition, test sets may be generated to target subjects of a specific age.

Tests may be generated having specific characteristics, e.g. memorability or vocabulary, based on a population group of the subject. For example the characteristics of tests may be set depending on the age of the subject, and/or whether they suffer a neurological disease, such as Alzheimer's disease. Characteristics of the test may depend on whether the subject is being screened for a specific neurological disease or monitored, having already been diagnosed.

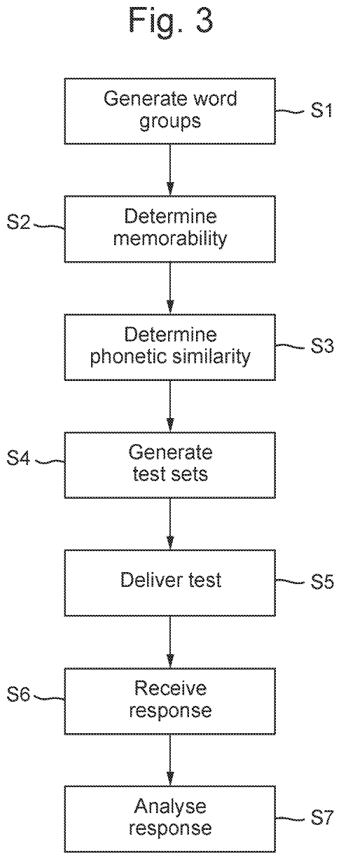

FIG. 3 is a flow chart showing an example process according to the disclosure. At S1 word groups are generated; at S2 memorability of word groups is determined; at S3 phonetic similarly of word groups is determined; at S4 test sets are generated based on the determined memorability and determined phonetic similarity; at S5 a test is delivered to a subject; at S6 a response from the subject is received; and at S7 the response is assessed.

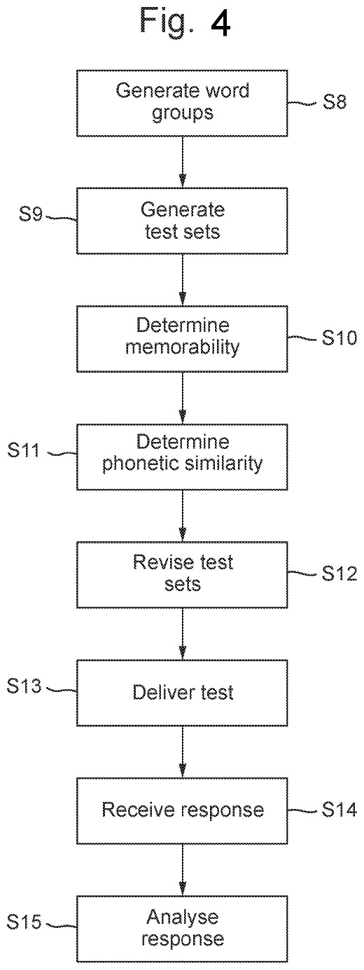

FIG. 4 is a flow chart showing another example process according to the disclosure. At S8 word groups are generated; at S9 test sets are generated; at S10 compound memorability of the sets is determined; at S11 phonetic similarly of the sets determined; at S12 test sets are revised based on the determined memorability and determined phonetic similarity; at S13 a test is delivered to a subject; at S14 a response from the subject is received; and at S15 the response is assessed.

In another example process, one or more of steps S2 and S3 may be performed in any order or simultaneously prior to test set generation and one or more of steps S10, S11 and S12 may be performed in any order or simultaneously after test set generation, and/or step S12 may be performed after both step S10 and step S11. In another example, only one of determining memorability and determining phonetic similarity may be performed (either for the word groups or the sets of word groups).

The above described system meets the need for generating sets of word-pairs in order facilitate high-frequency repeat testing of episodic memory deployed via automated voice platforms that employ speech recognition technology. This is particularly useful when testing the efficacy of treatments designed to slow or stop cognitive decline from neurological disease, where there is a great need for sensitive instruments that can detect subtle protective effects over the course of 18 to 24 months.

It should be understood that variations of the above described examples are possible without departing from the spirit or scope of the invention as defined by the claims.

1. A system for assessing the neurological function of a subject, comprising:
a database storing a plurality of word groups, said word groups comprising two or more words;
a word group memorability determining part for determining memorability of the word groups based on one or more characteristics of the words in the word groups;
a test set generating part for generating a plurality of sets of word groups, based on the memorability of the word groups; and
a test delivering part for delivering test to the subject in the form of at least one set of the plurality of sets of word groups for recall by the subject.

2. The system of claim 1, wherein the one or more characteristics comprises a measure of semantic relatedness between words in the word group, a higher semantic relatedness being associated with a higher memorability.

3. The system of claim 2, wherein sematic relatedness is measured by representing words as vectors using a machine learning algorithm and measuring cosine distance between said vectors.

4. The system of any preceding claim, wherein the one or more characteristics comprises a measure of word-frequency of words in the word group, a higher word-frequency being associated with a higher memorability.

5. The system of claim 4, wherein word-frequency is attributed to a word based on a look-up table comprising pre-stored a word-frequency for each word.

6. The system of any preceding claim, wherein the one or more characteristics comprises a measure of word-concreteness of words in the word group, a higher word-concreteness being associated with a higher memorability.

7. The system of claim 6, wherein word-concreteness is attributed to a word based on a look-up table comprising pre-stored a word-concreteness for each word.

8. The system of any preceding claim, wherein the one or more characteristics comprises the position of a word in the word group.

9. The system of any preceding claim further comprising a phonetic similarity determining part for determining the phonetic similarity between words in the word groups, wherein the test set generating part generates the plurality of sets of word groups based on the phonetic similarity between words in the word groups.

10. The system of claim 9, wherein the phonetic similarity is below a predefined threshold, such that the words are phonetically dissimilar.

11. The system of claim 10, wherein phonetic similarity is measured by representing the words phonetically and measuring an edit distance between said phonetic representations.

12. The system of any preceding claim, wherein the test delivering part delivers the at least one set of word groups aurally.

13. The system of any preceding claim, further comprising:
a response receiving part for receiving a response from the subject attempting to recall the delivered at least one set of word groups.

14. The systems of claim 13, wherein the response receiving part is configured to receive spoken responses.

15. The system of claim 14, further comprising a speech analysis part for determining a textual response from the spoken response.

16. The system of any one of claims 13 to 15, further comprising:
a response assessment part for comparing the response from the subject to the delivered at least one set of word groups.

17. The system of claim 16, wherein the response assessment part compares a textual response from the subject to the delivered at least one set of word groups.

18. The system of any preceding claim, wherein the system is configured to deliver at least two tests with predetermined time intervals between said tests, wherein the sets of word groups of the delivered tests are different and the memorability of the sets of word groups of the delivered tests satisfy a predetermined relationship.

19. The system of claim 18, wherein the predetermined relationship is that the tests have the same or similar memorability.

20. The system of claim 19, wherein the predetermined relationship is that consecutive tests have an incrementally lower memorability or an incrementally higher memorability.

21. A system for assessing the neurological function of a subject, comprising:
a database storing a plurality of word groups, said word groups comprising two or more words;
a phonetic similarity determining part for determining the phonetic similarity between words in the word groups,
a test set generating part for generating a plurality of sets of word groups based on the phonetic similarity between words in the word groups; and
a test delivering part for delivering test to the subject in the form of at least one set of the plurality of sets of word groups for recall by the subject.

22. The system of claim 21, wherein the sets are generated such that the phonetic similarity between words is below a predefined threshold, such that the words are phonetically dissimilar.

23. The system of any preceding claim comprising a word group generating part for generating the plurality of word groups from a base list of words and storing in said database.

24. A computer implemented method for assessing the neurological function of a subject, comprising:
storing a plurality of word groups, said word groups comprising two or more words;
determining memorability of the word groups based on one or more characteristics of the words in the word groups;
generating a plurality of sets of word groups, based on the memorability of the word groups; and
delivering test to the subject in the form of at least one set of the plurality of sets of word groups for recall by the subject.

25. A computer implemented method for assessing the neurological function of a subject, comprising:
reading a plurality of word groups stored in a memory, said word groups comprising two or more words;
determining memorability of the word groups based on one or more characteristics of the words in the word groups;
generating a plurality of sets of word groups, based on the memorability of the word groups; and
delivering test to the subject in the form of at least one set of the plurality of sets of word groups for recall by the subject.

26. A computer implemented method for assessing the neurological function of a subject, comprising:
storing a plurality of word groups, said word groups comprising two or more words;
determining the phonetic similarity between words in the word groups,
generating a plurality of sets of word groups based on the phonetic similarity between words in the word groups; and
delivering test to the subject in the form of at least one set of the plurality of sets of word groups for recall by the subject.

27. A computer implemented method for assessing the neurological function of a subject, comprising:
reading a plurality of word groups stored in a memory, said word groups comprising two or more words;
determining the phonetic similarity between words in the word groups,
generating a plurality of sets of word groups based on the phonetic similarity between words in the word groups; and
delivering test to the subject in the form of at least one set of the plurality of sets of word groups for recall by the subject.

28. A computer system comprising at least one processor and at least one memory configured to implement assessing the neurological function of a subject:
the at least one memory comprising a database configured to store a plurality of word groups, said word groups comprising two or more words;
the at least one processor comprising a word group memorability determining part configured to determine memorability of the word groups based on one or more characteristics of the words in the word groups;
the at least one processor comprising a test set generating part configured to generate a plurality of sets of word groups, based on the memorability of the word groups; and
the at least one processor comprising a test delivering part configured to deliver test to the subject in the form of at least one set of the plurality of sets of word groups for recall by the subject.

29. A non-transitory computer-readable medium storing code which, when executed by at least one processor of a computer system, causes the system to implement assessing the neurological function of a subject, comprising:
reading at least one memory of the computer system comprising a database storing a plurality of word groups, said word groups comprising two or more words;
a word group memorability determining part of the at least one processor determining memorability of the word groups based on one or more characteristics of the words in the word groups;
a test set generating part of the at least one processor generating a plurality of sets of word groups, based on the memorability of the word groups; and
a test delivering part of the at least one processor delivering a test to the subject in the form of at least one set of the plurality of sets of word groups for recall by the subject.

30. A computer system comprising at least one processor and at least one memory configured to implement assessing the neurological function of a subject:
the at least one memory comprising a database configured to store a plurality of word groups, said word groups comprising two or more words;
the at least one processor comprising a phonetic similarity determining part configured to determine the phonetic similarity between words in the word groups,
the at least one processor comprising a test set generating part configured to generate a plurality of sets of word groups based on the phonetic similarity between words in the word groups; and
the at least one processor comprising a test delivering part configured to deliver a test to the subject in the form of at least one set of the plurality of sets of word groups for recall by the subject.

31. A non-transitory computer-readable medium storing code which, when executed by at least one processor of a computer system, causes the system to implement assessing the neurological function of a subject, comprising:
reading at least one memory comprising a database configured to store a plurality of word groups, said word groups comprising two or more words;
a phonetic similarity determining part of the at least one processor determining the phonetic similarity between words in the word groups,
a test set generating part of the at least one processor generating a plurality of sets of word groups based on the phonetic similarity between words in the word groups; and
a test delivering part of the at least one processor delivering a test to the subject in the form of at least one set of the plurality of sets of word groups for recall by the subject.
